(19) United States
(12) Patent Application Publication
(10) Pub. No.: US 2026/0315173 A1
(43) Pub. Date: Oct. 15, 2026

(54) DEVICES, SYSTEMS, AND METHODS FOR CONTROLLING POWERED SURGICAL INSTRUMENTS

(71) Applicants: UNIVERSITY OF PITTSBURGH - OF THE COMMONWEALTH SYSTEM OF HIGHER EDUCATION, Pittsburgh, PA (US); UPMC, Pittsburgh, PA (US)

(72) Inventors: David H. Chi, Pittsburgh, PA (US); Therlking Dervil, Naples, FL (US); Jose P. Zevallos, Pittsburgh, PA (US); Benjamin Ratchford, Ebensburg, PA (US); Megan Johns, Ellwood City, PA (US); Paul Lecker, Burgettstown, PA (US)

(21) Appl. No.: 19/484,556

(22) PCT Filed: May 10, 2024
(86) PCT No.: PCT/US2024/028816

§ 371 Date: Nov. 14, 2025

Related U.S. Application Data
(60) Provisional application No. 63/466,417, filed on May 15, 2023.

(51) Int. Cl. A61B 90/00; A61B 17/00; A61B 17/02; A61B 18/00; A61B 18/12; A61B 34/20; A61B 34/30

(52) U.S. Cl. CPC A61B 90/04; A61B 18/12; A61B 34/30; A61B 2017/00022; A61B 17/02; A61B 2018/00327; A61B 2018/00595; A61B 2018/00642; A61B 2018/00702; A61B 2034/2072

(57) ABSTRACT

A system for performing a surgical procedure with a powered surgical device which is configured to be connected to a power source to power a tissue-interacting element of the powered surgical device, includes electronic circuitry configured to be placed in connection with the powered, surgical device and a sensor system in communicative connection with the electronic circuitry. The sensor system is configured to measure values of one or more variables related to a risk of injury to non-targeted tissue over time during the surgical procedure. The electronic circuitry is configured to control the tissue-interacting element as a function of the measured values of the one or more variables.

### CROSS-REFERENCE TO RELATED APPLICATIONS

This application claims benefit of U.S. Provisional Patent Application Ser. No. 63/466,417, filed May 15, 2023, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

A variety of powered surgical instruments or devices are used in various surgical procedures. Such powered surgical tools enhance surgeon performance and may provide significant improvements in outcome.

Electrocautery, for example, is a common technique in surgery to achieve results such as hemostasis, surgical dissection, or reduction in risk of infection. In electrocautery, a surgeon uses a device which converts electrical energy to heat which is used to heat targeted tissue. In current practice, an electrocautery device such as an electrocautery surgical pen or wand is connected directly to a current-generating system. Before the surgery, the surgeon places a grounding pad on the patient's body to protect the patient from potential harmful effects of the electric current. The electric current does not enter the body during surgery. Only the heated tip of a probe of the electrocautery device comes into contact with tissue. The heat tip or tissue-interacting element reaches a sufficiently high temperature to seal or remove the tissue it contacts.

Although electrocautery is a very common procedure, electrocautery has been associated with a number of significant problems. For example, catastrophic airway fires can occur in high-oxygen situations. Moreover, inadvertent burns of non-targeted, surrounding tissue (for example, bums of the lip or other facial tissue in otolaryngological surgeries such as tonsillectomies) may occur.

High-speed surgical drills are used in many surgical procedures (for example, neurosurgical, craniofacial, spinal, ENT, and orthopedic procedures) to quickly and accurately remove hard tissue. High-speed saws/microsaws are similarly used in a variety of surgical procedures. Like electrocautery devices, high speed surgical drills and saws can cause significant damage when the tissue-interacting element thereof (that is, a drill bit or a saw blade, respectively) make contact with non-targeted tissue. Misdirection or misapplication of powered surgical devices including tissue interacting elements which do not physically contact tissue (for example, laser-emitting surgical devices) can also cause significant damage to non-targeted tissue.

In addition to injury arising from interaction with non-targeted tissue, surgical instruments or devices that are operated at high temperature (for example, electrocautery devices) pose a risk of fire, particularly in a high-oxygen environment.

### SUMMARY

In one aspect, a system for performing a surgical procedure with a powered surgical device which is configured to be connected to a power source to power a tissue-interacting element of the powered surgical device, includes electronic circuitry configured to be placed in connection with the powered surgical device and a sensor system in communicative connection with the electronic circuitry. The sensor system is configured to measure values of one or more variables related to a risk of injury to non-targeted tissue over time during the surgical procedure. The electronic circuitry is configured to control the tissue-interacting element as a function of the measured values of the one or more variables. The electronic circuitry may be further configured to be placed in communicative connection with the power source and to control the tissue-interacting element via adjustment of power from the power source to the tissue-interacting element. In a number of embodiments, the electronic circuitry includes a processor system and a memory system in communicative connection with the processor system, the memory system having one or more algorithms stored therein, the one or more algorithms being executable by the processor system to analyze the measured values of the one or more variables to determine if one 2 or more thresholds have been met in determining the risk of injury to non-targeted tissue. The electronic circuitry may be configured to determine if a threshold associated with at least one of the one or more variables has been exceeded.

In a number of embodiments, at least one of the one or more variables is related to (i) a state of an environment in which the powered surgical device is operating during the surgical procedure or (ii) a state of position of the tissue-interacting element of the powered surgical device during the surgical procedure. In general, the phrase “state of position” refers to position in space, orientation in space, and variable derived from at least one of position in space and orientation in space. The at least one of the one or more variables may, for example, be related to the state of position of the powered surgical device and provide data regarding (a) a position of the tissue-interacting element, (b) a rate in change of the position of the tissue-interacting element, (c) a trajectory of the tissue-interacting element, or (d) an orientation of the tissue-interacting element. The electronic circuity may, for example, be configured to reduce power to the tissue-interacting element if the position of the tissue-interacting element is determined to be outside of a predetermined boundary.

In a number of embodiments, the sensor system includes one or more localization sensors. The one or more localization sensors may, for example, include at least one of a proximity sensor, a sensor for a target gas, or an imaging system sensor of an imaging system.

The system may further include a localization system comprising at least one of (i) a frame configured to be positioned adjacent to at least a portion of a surgical region of interest or (ii) one or more fiducials configured to be a reference for an imaging system sensor of an imaging system. The frame may, for example, be a component of a surgical retractor. In a number of embodiments, the frame includes one or more components attached thereto which interact with one or more components on the powered surgical device to localize the position of the tissue-interacting element of the powered surgical device. One of the frame or the powered surgical device may, for example, include one or more localization sensors and the other of the frame or the powered surgical device may include one or more components which interact with the one or more localization sensors.

In a number of embodiments, the frame has a plurality of energy sources attached thereto and a plurality of energy receivers attached thereto. The powered surgical device may include one or more areas of material which is/are configured to reflect the energy from one or more of the plurality energy sources to the one or more of the plurality of energy receivers. The plurality of energy sources emit energy in at least two different directions, wherein energy emitted in each direction is distinguishable from energy emitted in another direction upon reflection to the plurality of energy receivers from the one or more area of material. In a number of embodiments, the plurality of energy sources emit energy in a first direction and in a second direction. The second direction may, for example, be approximately orthogonal to the first direction.

In a number of embodiments, the one or more localization sensors include one or more imaging system sensors of an imaging system. In a number of embodiments, the surgical procedure is a robotic assisted surgical procedure. The system may, for example, include a selection system via which a user can define a predetermined boundary or boundaries.

In a number of embodiments, the powered surgical device is a surgical drill device, a surgical saw device, an electrocautery device, laser emitting device, or a microdebrider device. In a number of embodiments, the powered surgical device is an electrocautery device. In a number of embodiments, the powered surgical device is an electrocautery device, the tissue-interacting element is an electrocautery probe and the frame is a component of an oral surgical retractor.

In a number of embodiments, at least one of the one or more variables relates to the state of the environment in which the powered surgical device is operating, and the sensor system comprises one or more sensors comprising at least one of a temperature sensor or a gas sensor. The gas sensor may, for example, include an oxygen sensor The electronic circuity may, for example, be configured to reduce power (including reducing power to zero or stopping power) to the powered tissue-interacting element if a predetermined oxygen concentration threshold is exceeded. In a number of embodiments, the powered surgical device is an electrocautery device.

One or more components of the system hereof may be configured to be added or retrofitted onto the powered surgical device. The electronic circuitry may, for example, be a component of a module configured to be placed in electrical connection with the powered surgical device.

In another aspect, a method for performing a surgical procedure with a powered surgical device which is configured to be connected to a power source to powered a tissue-interacting element of the powered surgical device, includes placing electronic circuitry in connection with the powered surgical device, measuring values of one or more variables related to a risk of injury to non-targeted tissue over time during the surgical procedure via a sensor system in communicative connection with the electronic circuitry, and controlling the tissue-interacting element as a function of the measured values of the one or more variables. The electronic circuitry may further be configured to be placed in communicative connection with the power source and to control the tissue-interacting element via adjustment of power from the power source to the tissue-interacting element. In a number of embodiments, the electronic circuitry comprises a processor system and a memory system in communicative connection with the processor system, the memory system having one or more algorithms stored therein, the one or more algorithms being executable by the processor system to analyze the measured values of the one or more variables to determine if one or more thresholds have been met in determining the risk of injury to non-targeted tissue.

At least one of the one or more variables are related to (i) a state of an environment in which the powered surgical device is operating during the surgical procedure or (ii) a state of position of the powered surgical device during the surgical procedure. In a number of embodiments, the at least one of the one or more variables is related to the state of position of the powered surgical device and provides data regarding (a) a position of the tissue-interacting element, (b) a rate in change of the position of the tissue-interacting element, (c) a trajectory of the tissue-interacting element, or (d) an orientation of the tissue-interacting element. The electronic circuity may, for example, be configured to reduce power to the tissue-interacting element if the position of the tissue-interacting element is determined to be outside of a predetermined boundary.

In a number of embodiments, the sensor system includes one or more localization sensors. The one or more localization sensors may include at least one of a proximity sensor, a sensor for a target gas, or an imaging system sensor of an imaging system.

As described above, the at least one the one or more variables may relate to the state of the environment in which the powered surgical device is operating. The sensor system may, for example, include one or more sensors comprising at least one of a temperature sensor or a gas sensor. The gas sensor may, for example, be an oxygen sensor. The electronic circuity may be configured to reduce power to the powered tissue-interacting element if a predetermined oxygen concentration threshold is exceeded. As described above, the powered surgical device is an electrocautery device.

The method may further be characterized as set forth elsewhere herein.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates schematically a representative embodiment of an electrocautery system hereof.

FIG. 1B illustrates schematically a block diagram of electrocautery systems hereof.

FIG. 2A illustrates schematically a top view of an embodiment of an oxygen (O₂) monitoring system which is attachable to an electrocautery device.

FIG. 2B illustrates schematically a side view of the O₂ monitoring system of FIG. 2A.

FIG. 2C illustrates schematically a bottom view of the O₂ monitoring system of FIG. 2A.

FIG. 3A illustrates schematically a top view of an embodiment of an oxygen (O₂) monitoring and proximity sensor system which is attachable to an electrocautery device.

FIG. 3B illustrates schematically a side view of the attachable system of FIG. 3A.

FIG. 3C illustrates schematically a bottom view of the attachable system of FIG. 3A.

FIG. 4A illustrates schematically a top view of an embodiment of an attachable O₂ monitoring system hereof which includes marking for cooperation with an inductive proximity sensor or sensors attached to the patient.

FIG. 4B illustrates schematically a side view of the system of FIG. 4A.

FIG. 5 illustrates schematically an embodiment of a mouth retractor system hereof including a proximity sensing system using infrared energy to sense proximity of an electrocautery device.

FIG. 6 illustrates schematically an embodiment of a mouth retractor system hereof including pattern of marking for cooperation with an inductive proximity sensing system attached to an electrocautery device.

FIG. 7 illustrates schematically an embodiment of a mouth retractor system hereof including inductive proximity sensors.

FIG. 8 illustrates schematically an embodiment of a retractor system hereof in operative connection with a patient's mouth for proximity sensing of the illustrated electrocautery device.

FIG. 9A illustrates schematically the mouth retractor system of FIG. 5 wherein infrared energy is schematically illustrated in broken lines passing between laterally positioned infrared emitter/receivers on the mouther retractor system.

FIG. 9B illustrates schematically the interaction of the mouth retractor system of FIG. 9A with an electrocautery device.

FIG. 10A illustrates schematically a mouth retractor system hereof including a pattern of markings (for example, spaced lines) on laterally positioned elements thereof.

FIG. 10B illustrates schematically the interaction of an electrocautery device including one or more inductive sensors positioned thereon with the mouth retractor system of FIG. 10A.

FIG. 10C illustrates a measure signal upon forward motion of the electrocautery device relative to the mouth retractor system of FIG. 10A.

FIG. 10D illustrates a measure signal upon rearward motion of the electrocautery device relative to the mouth retractor system of FIG. 10A.

FIG. 11A illustrates schematically an embodiment of an electrocautery device hereof including a pattern of markings (for example, lines) thereon.

FIG. 11B illustrates schematically the interaction of the electrocautery device of FIG. 11A with a retractor system such as that illustrated in FIG. 7, which includes one or more induction proximity sensors in lateral elements thereof.

FIG. 12A illustrates schematically another embodiment of a localization system hereof for use in connection with an electrocautery device.

FIG. 12B illustrates schematically representative patterns of energy transmission in a localization system used in connection with a frame.

FIG. 13 illustrates schematically another embodiment of a localization system hereof wherein localization is performed via an imaging system.

### DESCRIPTION

The present devices, systems, and methods, along with the attributes and attendant advantages thereof, will best be appreciated and understood in view of the following description taken in conjunction with any accompanying drawings.

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a predetermined boundary” includes a plurality of such predetermined boundaries and equivalents thereof known to those skilled in the art, and so forth, and reference to “the predetermined boundary” is a reference to one or more such predetermined boundaries and equivalents thereof known to those skilled in the art, and so forth. Recitation of ranges of values herein are merely intended to serve as a shorthand method of referring individually to each separate value falling within the range. Unless otherwise indicated herein, each separate value as well as intermediate ranges are incorporated into the specification as if it were individually recited herein. All methods described herein can be performed in any suitable order unless otherwise indicated herein or otherwise clearly contraindicated by the text.

The terms “electronic circuitry”, “circuitry” or “circuit,” as used herein include, but is not limited to, hardware, firmware, software, or combinations of each to perform a function(s) or an action(s). For example, based on a desired feature or need. a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other types of executable instructions. It will be appreciated by one of ordinary skill in the art that the form of software is dependent on, for example, requirements of a desired application, the environment it runs on, or the desires of a designer/programmer or the like.

In a number of embodiments, the devices, systems, and methods hereof provide improved safety and/or efficacy in the use of powered surgical instruments such as electrocautery devices, high-speed drills, saws/microsaws, debrider/microdebrider, laser-emitting surgical instruments (for example, laser ablation devices) etc. which include a powered tissue-interacting element (that is, an electrocautery probe/electrode, a drill bit, a saw blade, laser energy source, debrider element, etc.) that effects or causes a change in tissue during a surgical procedure. In currently available surgical instruments or devices that are operated under power other than manual power (for example, under electrical power, pneumatic power etc.), the surgeon manually controls the motion and position of the surgical device and its tissue-interacting element (either via direct physical control of the device or via a robotic system). In robotic assisted surgical procedures, the surgeon controls one or more robotic arms while at a control center to control a surgical device. Further, the surgeon controls the power supplied to the tissue-interacting element of such powered surgical instruments. Although surgeons are highly trained and surgery occurs under strict controls, contact with (and attendant damage to) non-targeted tissue (that is, tissue which is not the target of a particular surgical procedure) may occur in the use of such powered surgical instruments.

In devices, systems, and methods hereof, sensor systems are used to detect one or more variables/conditions that are associated with a predetermined or predefined risk condition. Such a risk condition may be defined as injury to the patient, which may further be defined as injury to tissue other than the tissue targeted for change via the surgical procedure (that is, “targeted tissue). Electronic circuitry hereof is configured to control the tissue-interacting element of a powered surgical device as a function of the measured values of the one or more variables/conditions (to, for example, reduce the risk injury to tissue other than the targeted tissue (that is, to non-targeted tissue). For example, various thresholds values for the one or one or more variable may be established for actuating such control. In a number of embodiments hereof, the one or more variables are measured which are related to (or a measure of) (i) a state of an environment in which the powered surgical device is operating during the surgical procedure or (ii) a state of position of the powered surgical device during the surgical procedure.

The one or more variables related to (or a measure of) a state of position of the tissue-interacting element of the powered surgical device may, for example, be related to (or a measure of) a position of the tissue-interacting element in space, a trajectory (or path) of motion of the tissue interacting element, an angle/orientation of the tissue-interacting element, or to a rate in change of position of the tissue interacting element. An angle/orientation of the tissue-interacting element may, for example, be particularly relevant in an energy-emitting surgical device such as a laser-energy-emitting surgical device.

Control of the tissue-interacting element may, for example, include control or adjustment of power to the tissue-interacting element. Control may additionally or alternatively include actuation of a braking mechanism in the case that the tissue-interacting element is a movable element (for example, a drill bit or a saw blade), or actuation of a blocking or shutter mechanism in the case that the tissue-interacting element is an energy-emitting element.

In a number of embodiments, a notification/alarm may be provided to the surgeon/operator of a surgical device hereof (for example, before or at the time the electronic circuitry begins to control the tissue-interacting element to reduce risk to non-targeted tissue, such as) if one or more of the one or more variables has reached a threshold value. For example, if the position of the tissue-interacting element is in the vicinity of a threshold position or predetermined boundary position/surface, if the tissue-interacting element is approaching such a boundary at a defined rate, or is on a predetermined trajectory, an alarm signal may be provided (for example, an audio, tactile, and/or visual signal).

As described above, in a number of embodiments hereof, the sensor system is in communicative connection with electronic circuitry that is configured to control power to a powered surgical device as a function of a measured value of the one or more variables. If the predetermined or predefined risk condition is determined via data from the sensor system, the power to the tissue-interacting element of the powered surgical device can be reduced (including stopped) via the electronic circuitry.

In a number of representative embodiments hereof, sensor systems hereof include one or more position, location or localization sensors which may, for example, function in operative connection with one or more components of a localization system to determine variables related to position of the tissue-interactive element of a powered surgical device relative to one or more defined boundaries. Power to a powered surgical device which presents a danger of injury to non-targeted tissue can be reduced if it is, for example, determined via measured data from the localization sensor(s) that the tissue-interacting element of the powered surgical device is outside of a defined boundary (for example, a two-or three-dimensional surface associated with a surgical volume or surgical region of interest).

In representative embodiments hereof in which a powered surgical instrument is operated at high temperature, a sensor system hereof may be used to determine one or more risk variable/conditions (for example, environmental variables/conditions in the vicinity of the tissue-contacting element) associated with fire. Power to the tissue-interacting element of such a powered surgical device can be reduced if it is, for example, determined via the sensor system that one or more predetermined threshold conditions have been measured.

In a number of embodiments hereof, the devices, systems, and methods hereof are discussed in connection with electrocautery devices. However, one skilled in the art will appreciate that the systems hereof may be used in connection with any powered surgical instrument which includes a powered tissue-interacting element.

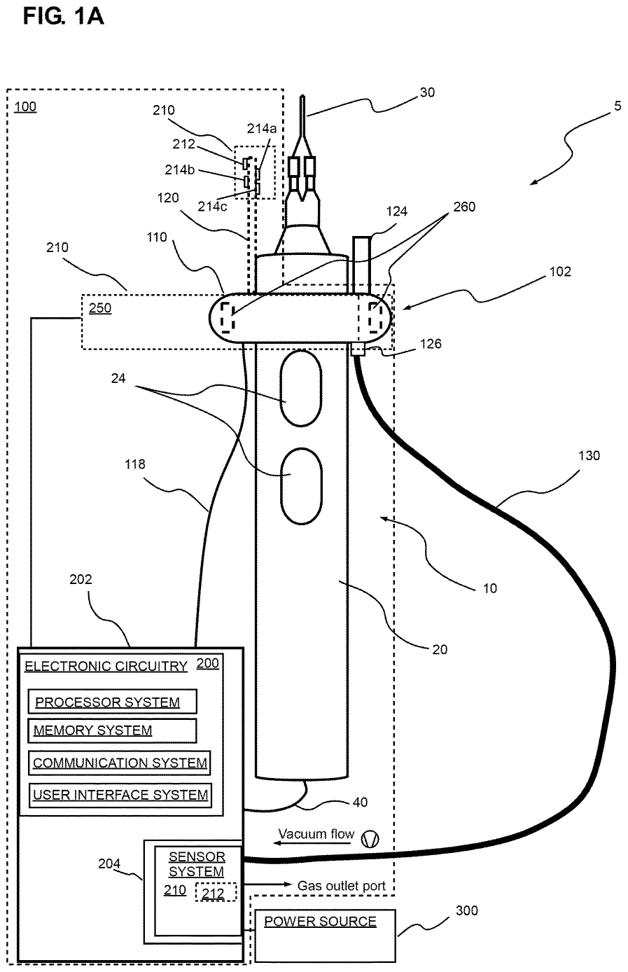

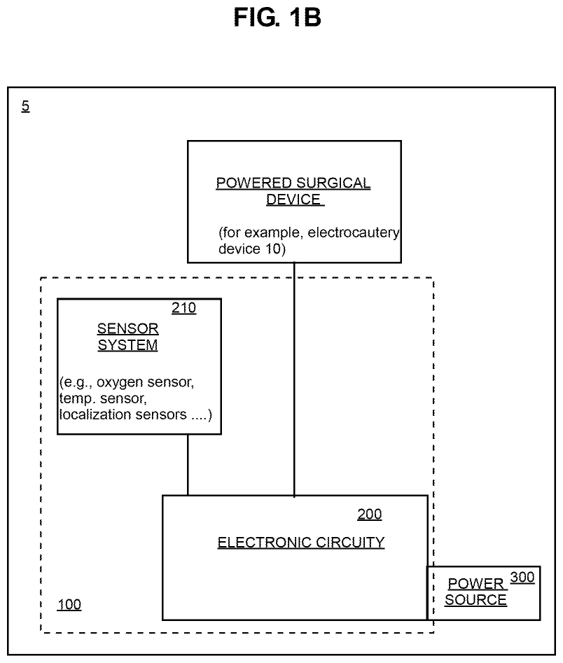

In that regard, a representative embodiment of a system 5 hereof including an electrocautery device is illustrated in FIG. 1A and 1B. Further description of representative embodiments of the devices, systems, and methods hereof (particularly in connection with otolaryngological surgeries involving electrocautery) is provided in connection with FIGS. 2A through 11B. One skilled in the art will appreciate that although otolaryngological surgeries involving electrocautery are discussed as representative example, the devices, systems, and methods hereof may be used in connection with any powered surgical procedure regardless of the location of the surgical region/volume of interest. Moreover, a number of representative embodiments of devices, systems, and methods hereof are described in connection with one or more add-on modules, systems or devices which is/are connectible to existing powered surgical instrument or devices (for example, electrocautery pens, wands etc.). One skilled in the art will appreciate that all or varying portions/components of such add-on modules, systems or devices can be readily incorporated or integrated into powered surgical instruments or devices.

FIG. 1A, illustrates a representative example of a commercially available electrocautery device 10 in operative connection with a system 100 hereof (which may include or be operatively connected to electronic circuitry 200). Electrocautery device 10 includes a body 20 which is used as a handle by the user. As known in the art, one or more actuators 24 (for example, buttons) may be included on body 20 which may be held by a user and may include manual control functionality for a surgeon to manually control power/current to a tissue-interacting element in the form of an electrocantery probe, electrode, or knife 30 positioned on a distal end of electrocautery device 10. Connecting circuitry/wiring 40 is provided (illustrated as extending from a distal end of body 20 of electrocautery device 10) to connect electrocautery device 10 to a power source 300. In current practice, powered surgical instruments or devices such as electrocautery surgical devices are connected directly to a power source/current-generating system.

In FIG. 1A, an add-on or attachable component or system 102 of system 100 is readily used in connection with currently available electrocautery devices 10 (for example, electrocautery wands or pens) which are used in surgeries such as tonsillectomies. As described, for example, in connection with FIGS. 1A through 2C, a body, base or interface 110 of system 102 may encompass a portion of body 20 of device 10 in a number of embodiments hereof In that regard, base 110 may include a passage 112 (see, for example, FIG. 2A) through which a forward section of electrocautery device 10 may be passed. A material or material layer 116 (for example, a resilient material such as an elastomeric polymeric material) may be present on an inner wall of passage 112 to assist in gripping electrocautery device 10. Additionally or alternatively, one or more cooperating (for example, interlocking) mechanical mechanisms (not shown) may be provided to system 100 and electrocautery device 10 to assist in forming a secure connection therebetween.

Representative system 102 may be at least partially integrated with (illustrated schematically in FIG. 1B) or be operatively connected to electronic circuitry 200. System 100, may, for example, provide functionality to control power/current to electrocautery probe 30 of device 10, regardless of the state of actuator(s) 24. Control of power/current to probe 30 may, for example, be used to reduce power to probe 30 when unsafe or dangerous conditions are detected or determined (for example, when probe 30 is determined to be at the boundary of or outside of a defined volume or region of interest of if a threshold risk of fire is determined). Reduction of stoppage of power to probe 30 very quickly (almost instantaneously) reduce the temperature of probe 30 to a temperature safe for tissue and below a temperature at which ignition can occur in a high-oxygen environment. In the illustrated embodiment, electronic circuitry 200 is in operative connection with system 100 via a wire and/or another electrical conductor 118. As described above, system 100 hereof may override the functionality of actuator(s) 24 upon detection of predetermined or defined conditions (associated with measured values of one or more variables via a sensor system 210). For example, various environmental conditions or variables in the vicinity of a surgical region of interest (that is, an area/volume or target area/volume of surgery such as the tonsils) or area of operation of probe 30 may be detected by one or more sensors of sensor system 210. Such sensors may be distributed in position (see, for example, FIG. 1A) and need not be in physical contact with device 10 or a component of system 100. Such environmental conditions may, for example, include gas level(s) and/or temperature.

A gas sensor 212 of sensor system 210 may, for example, be positioned near the surgical region of interest (for example, on an extending member 120 which extends from base 110). Alternatively or additionally, a port or conduit 124 may be formed in or extend from base 110 to draw gas from the vicinity of the surgical region of interest to be transported to one or more gas sensors of sensor system 210 positioned remote from base 110 via a conduit 130 attachable to an attachment port 128. Port 124 and attachment port 128 are, for example, placed in fluid connection via a conduit 126 passing through base 110. One or more such sensors may, alternatively or additionally, be operatively connected to a component that is separate from, but in communication with, system 100. For example, one or more of the sensors of sensor system 210 may be attached to other surgical equipment (for example, a mouth retractor system as discussed further below) or attached directly to patient tissue. In addition to one or more gas sensors, other sensors 214a, 214b, 214c . . . (such as a temperature sensor) may be included in sensor system 210. Communication between sensors (which may be positioned remote from electronic circuitry 200) and electronic circuitry 200 may be in a wired or wireless manner.

In one embodiment, a negative pressure or vacuum system facilitates continuous gas/air flow from the surgical region of interest (for example, within the cavity of a patient's mouth or other cavity/volume) via port 124 and conduit 130 to an air/gas sampling system positioned, for example, within a small chamber 204 within a housing 202 (represented schematically in FIG. IA) for electronic circuitry 200. Small chamber 204 positioned, for example, within housing 202 for electronic circuitry 200 may hold oxygen sensor 212 as described above to provide live of real-time measurements/readings of the oxygen (O₂) concentration in the chamber (which may be calibrated to or extrapolated to represent the O₂ concentration in the mouth/region of surgical interest). Oxygen sensor 212 (for example, an electrochemical oxygen sensor as known in the sensor arts) may, for example, be used to detect oxygen concentration to enable analysis via electronic circuitry 200 to determine if a concentration threshold of oxygen concentration (for example, in cases in which oxygen enriched gas is being provided to the patient) has been exceeded such that a risk of fire exists. The sampled air may flow via vacuum driven flow into the chamber and out of a gas outlet port.

Sensor system 210 may include localization sensors 250 hereof (see, for example, FIGS. LA and 1B, in which localization sensors 250 are illustrated schematically), which may be used to localize, determine, or attribute variable related to the position of surgical probe 30 (for example, relative to or within a surgical region of interest). For example, in a number of embodiments, proximity sensors are used in determining the position of surgical probe 30 over time during a surgical procedure. One or more proximity sensors may, for example, be attached to the electrocautery device or attached to a component other than the electrocautery device positioned adjacent to or within the surgical region of interest. In one embodiment, one or more proximity sensors 260 (see FIG. 1A) are attached or integrated into base 110. Alternatively, proximity sensors may be integrated into a device positioned at or near the surgical region of interest (for example, on a cheek retractor system or mouthpiece). Proximity sensors 260 may, for example, provide live or real-time readings or measurements of when probe 30 of electrocautery device 10 is near tissue of the surgical region of interest as, for example, defined by a boundary surface. An element which selectively interacts with proximity sensor(s) 260 may, for example, be positioned near the surgical region of interest or attached/applied to tissue in the vicinity thereof. In the case that a proximity sensors is integrated into a device positioned at or near the surgical of region of interest such as upon a cheek retractor mouthpiece in the case of electrocautery surgery within the oral cavity, the proximity sensor provides live or real-time readings or measurements of when probe 30 of electrocautery device 10 is near the positioned device.

As known in the art, localization systems based upon proximity sensors may, for example, use detection of energy such as electromagnetic energy (over the spectrum thereof—for example, radio waves, light waves, etc.), heat energy, or sound energy to determine proximity. In general “proximity sensors” are sensors that perform non-contact detection in comparison to sensors that detect objects by physically interacting them. Proximity sensors convert information on the movement or presence of an object into an electrical signal. Representative types of detection systems include systems that use the eddy currents that are generated in metallic sensing objects by electromagnetic induction, detection systems that detect changes in electrical capacity when approaching the sensing object, and detection systems that use magnets and reed switches.

Imaging systems (that is, medical imaging system, which may use various forms of energy to generate images—such a tomography imaging, ultrasound imaging, etc.) may also be used in systems hereof to determine the state of or related to the position of probe 30 of electrocautery device 10. Photographic/camera imaging system (for example, video camera systems) may also be used as localization sensors.

Localization systems hereof, which are further discussed below, may be used to determine if electrocautery probe 30 is within, at a predetermined boundary of, or outside of the surgical region of interest. If probe 30 is outside of a boundary of surgical region of interest (or outside a boundary of defined volume within a surgical region of interest), power/current to probe 30 may be reduced (including, turned off) to reduce or eliminate the risk of injury to tissue outside of the surgical region of interest (that is, non-targeted tissue).

Electronic circuitry 200 may, for example, include a controller or control system to control operation of device 10 and to analyze or interpret the responses/data from at least one of (i) various sensors of sensor system 210 and (u) data from the localization system. As illustrated, for example, in FIG. 1A, electronic circuitry 200 may, for example, include a processor system (for example, including one or more microprocessors) in operative connection with a memory system. As known in the art, elements of electronic circuitry 200 may be incorporated in one or more printed circuit boards. One or more software algorithms may be stored in the memory system which is/are executable by the processor system to control/operate electrocautery device 10 as described herein. A user interface system (including, for example, a display, a speaker, a tactile system, various input/output systems, etc.) may also be placed in operative or communicative connection with the processor system. A communication system may be in operative or communicative connection with processor system for wired and/or wireless communication to other devices/systems. Power source 300 (for example, a battery system, line power, or an inductive energy transfer system) provides power for electronic circuitry 200 and electrocautery device 10.

In a number of embodiments hereof, the risk of injury to a patient is reduced or eliminated by reducing or cutting the power/current to an electrocautery probe if there is a risk of fire or if the electrocautery probe is outside of one or more predetermined boundaries such that non-targeted tissue might be damaged upon contact with electrocautery probe 30. Once again, systems hereof for power/current control may readily be provided as add-on modules/systems for existing electrocautery (or other powered surgical) devices or at least partially integrated in such devices.

In a number of embodiments hereof, electronic circuitry 200 operates in the manner of a power/current intervening, a power/current controlling system, or power control module which is electrically connected between power source or current generating system 300 and the powered surgical device. Electronic circuitry 200 may, for example, be operated as an intermediate current modulator to reduce or eliminate heating of electrocautery probe 30 or other powered tissue-interacting element (via control of power/current supplied thereto) on the basis of measured/determined conditions (for example, fire risk or localization/position) and predetermined thresholds for such conditions to reduce the risk of injury to non-targeted tissue.

In that regard, measurements/data from one of or both of oxygen sensor(s) 212 (and/or other sensor of sensor system 210) and localization sensor(s) 250 in certain embodiments hereof are processed or analyzed by the processing system of electronic circuitry 200. Electronic circuitry 200 uses, for example, software-based input parameters to determine whether signal/variable value thresholds have been met. Based upon the determined or measured response, electronic circuitry 200 controls the power/current supplied to probe 30 of electrocautery device 10. In such a manner, electrocautery device 10 (and other powered surgical devices) may function only when the oxygen levels are sufficiently low to prevent airway fires and/or when the powered tissue-interacting element thereof is located within one or more determined or defined boundaries of the desired surgical field to prevent injury (for example, burns etc.) of the surrounding/non-targeted tissue (including skin).

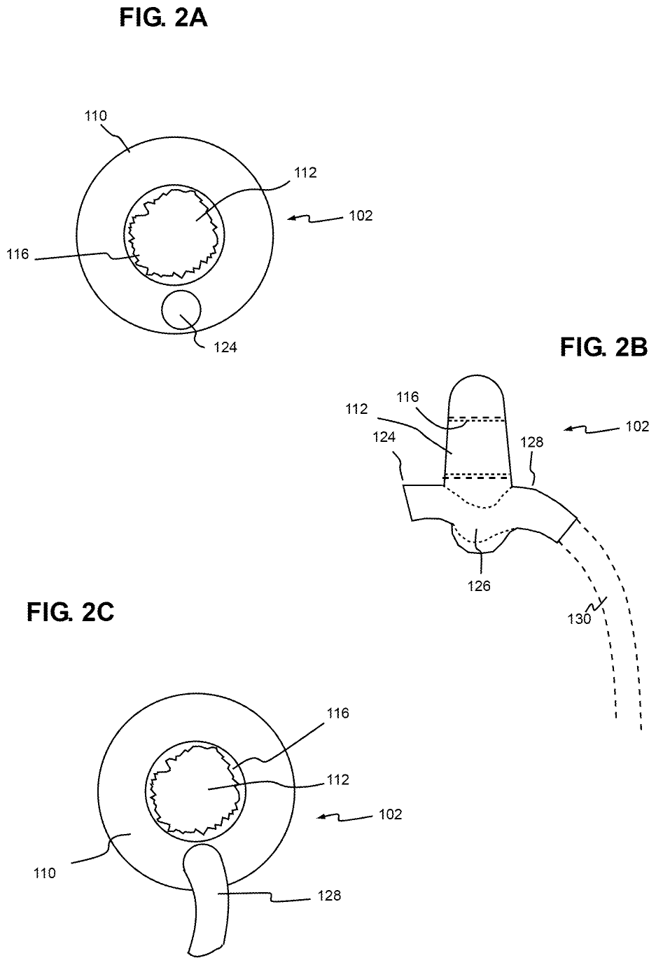

FIGS. 3A through 3C illustrate another embodiment of an add-on or retrofitting system 102a. System 102a is, in a number of respects, similar in function and design to system 102. Elements of system 102a are identified or referenced similarly to like components of system 102 with the addition of the designation “a” to the end of the reference. As described in connection with system 102, base or interface 110a of system 102a may encompass a portion of body 20 of device 10 in a number of embodiments hereof. In that regard, base 110a includes a passage 112a through which a forward section of electrocautery device 10 (or other electrocautery device) may be passed. A material or material layer 116a (for example, a resilient material such as an elastomeric polymeric material) may be positioned an inner wall of passage 112a to assist in gripping electrocautery device 10 as well as assist in appropriately sizing passage 112a to a particular electrocautery device. In the embodiment of system 100a, a protective sleeve or tube 134a is attachable to connection port 128a, through which a conduit or tubing 130a passes which functions as described from conduit 130.

In the illustrated embodiment of FIGS. 3A through 3C, one or more proximity sensors 260a (one in the illustrated embodiment) such as an inductive proximity sensor is/are positioned within one or more compartments 111a (one in the illustrated embodiment) formed on base 110a. A sensor wire 246a extends from sensor 260a, through body 110a, and through protective tubing 134a to, for example, electronic circuitry 200.

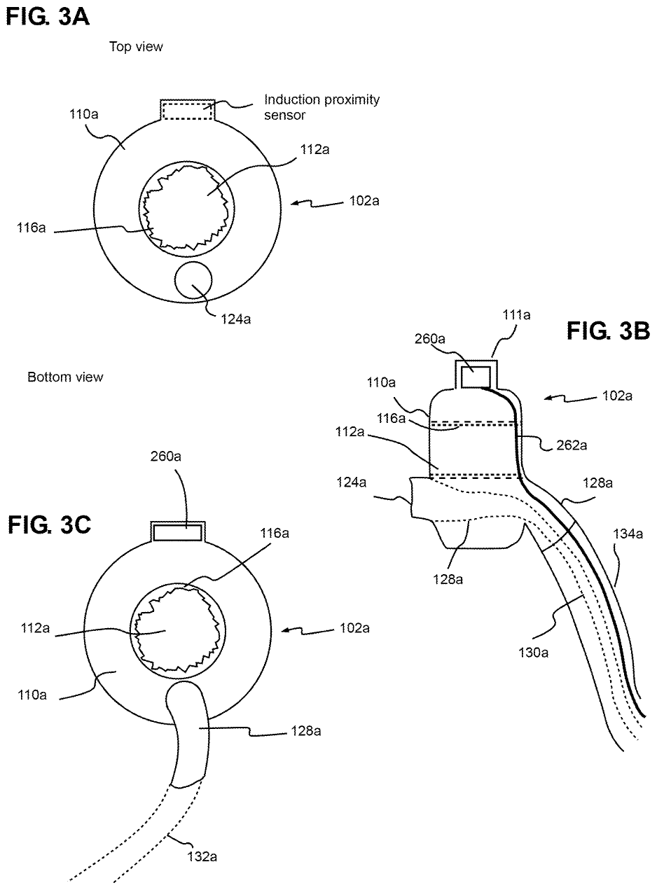

FIGS. 4A and 4B illustrate another embodiment of an add-on or retrofitting system 102b. System 102b is, in a number of respects, similar in function and design to systems 102 and 102a. Elements of system 102b are identified or referenced similarly to like components of systems 102 and 102a with the addition of the designation “b” to the end of the reference. In the illustrated embodiment of FIGS. 4A and 4B, one or more marking areas such as lines 272b (two in the illustrated embodiment) form a pattern 270b on an outer side wall of base 110b to interact with a sensor system such as an inductive proximity sensor system separate from system 102b as described further below.

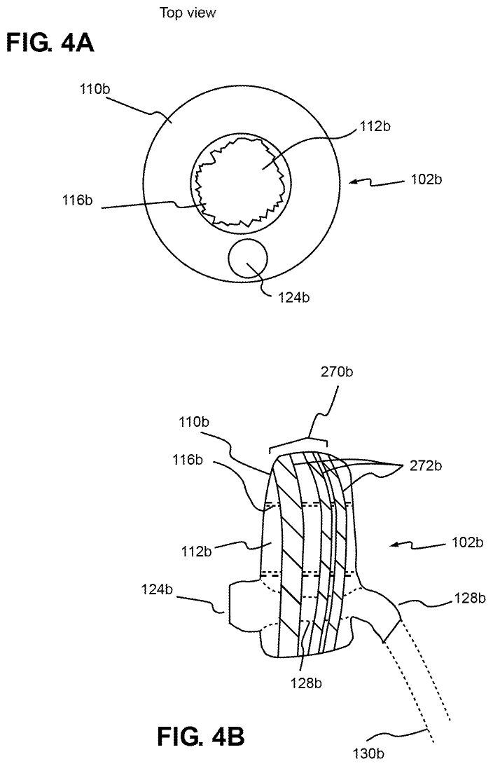

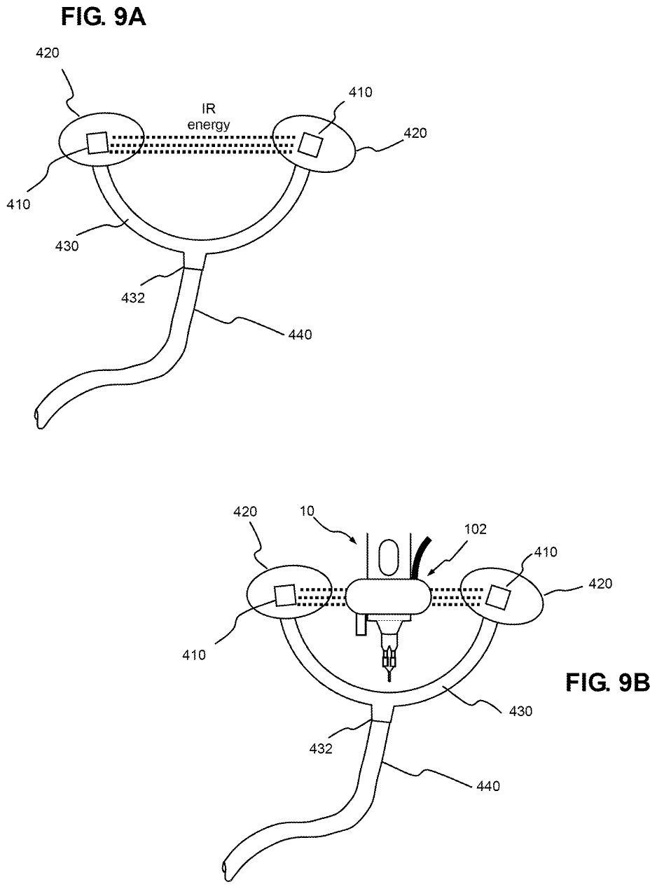

FIGS. 5 through 8 illustrate (schematically) embodiments of mouth retractor systems (sometime referred to as a cheek retractor mouthpieces or simply retractors) for use in localization systems for a powered surgical devices such as electrocautery devices. Such localization systems may interact with localization sensors or sensor systems 250 as described herein. As known in the art, such mouth retractor systems typically include at least lateral (left-and right-side) flanges to abut the lateral edges of the mouth. FIG. 8 illustrates mouth retractor system 400 of FIG. 5 in operative connection with a patient's mouth and electrocautery device 10 positioned to enter the patient's mouth and interact with the localization system components of mouth retractor system 400. In the embodiments of FIGS. 5 through 7, one or more components or elements that cooperate with a powered surgical device such as an electrocautery device to achieve localization are integrated with or connected to the mouth retractor system. In that regard, the retractor system functions as a frame for positioning such localization components or elements. Similar localization frames (for example, integrated with retractors/retractor system or as separate devices) may be used in surgeries in regions of interest other than oral and/or throat surgeries.

In FIG. 5, mouth retractor system 400 is a component of a localization system including infrared (IR) devices 410 positioned on each lateral side thereof which may be IR emitter and/or receivers (sensors). IR devices 410 may, for example, be positioned on or integrated with lateral side components 420 (for example, flanges) of retractor system 400. Wires 412 may, for example, pass through a frame 430 of mouth retractor system 400, through an end member 432 and into a protective conduit or tube 440 to, for example, place IR devices 410 in electrical connection with electrical circuitry 300 or other electrical circuitry.

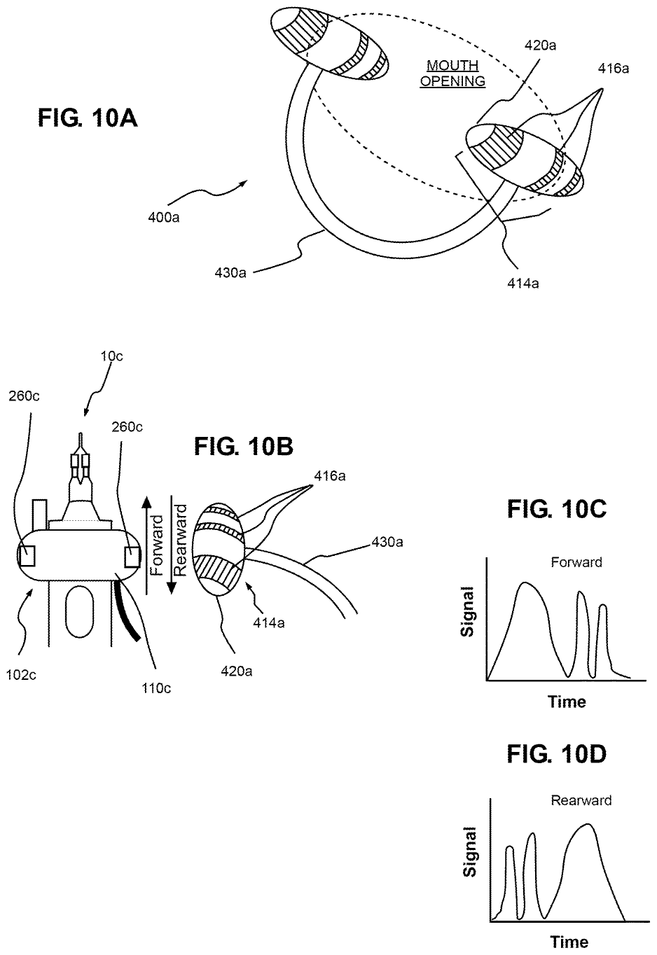

FIGS. 9A and 9B illustrate the function of mouth retractor system 400 of FIG. 5. FIG. 9A illustrates the transmission of IR energy between IR devices 410 as represented by broken lines extending between IR devices 410. FIG. 9B illustrates disruption of the transmission of IR energy between IR devices 410 by electrocautery device 10, enabling localization of electrocautery device 10. Mouth retractor system 400 may be positioned at or define the opening into a surgical region of interest corresponding to the oral cavity. Multiple IR devices 410 may be positioned at different depths (in the oral cavity) on mouth retractor system 400 (for example, on lateral flanges thereof) to provide further (for example, depth) information on the localized position of electrocautery device 10. In the case of IR devices 410 as described above, no components of the localization system need be placed on electrocautery device 10 or other electrocautery devices. One or more portions of electrocautery device 10 simply interrupt the transmission of energy (for example, IR energy) between an energy emitter and an energy receive/localization sensor of sensor system 210 hereof.

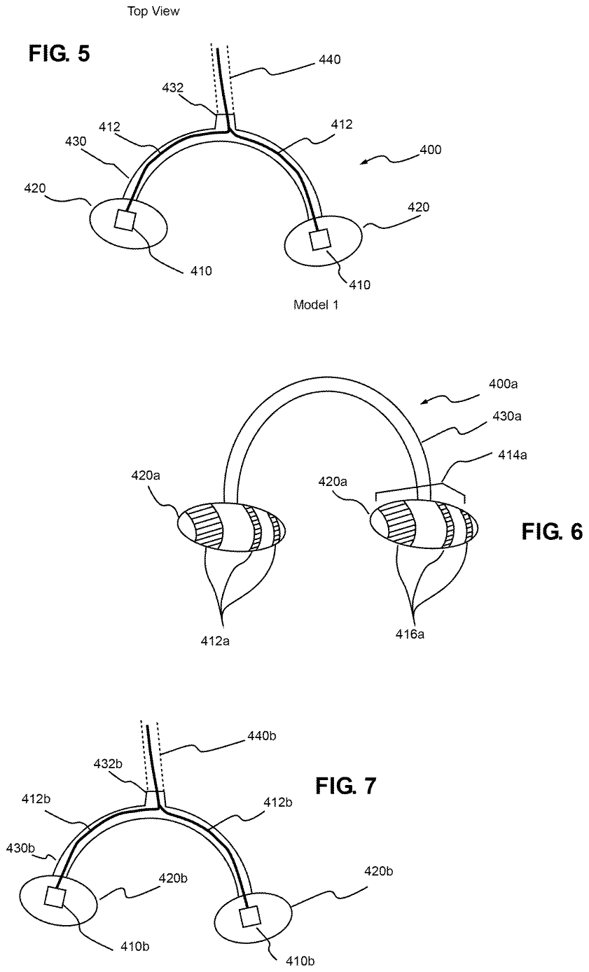

FIG. 6 illustrates another embodiment of a mouth retractor system 400a hereof. Mouth retractor system 400a is, in a number of respects, similar in function and design to mouth retractor system 400. Elements of mouth retractor system 400a are identified or referenced similarly to like components of system 400 with the addition of the designation “a” to the end of reference. Mouth retractor system 400a includes components of a localization system including one or more detectable areas or a pattern of such detectable areas 414a. Detectable pattern 414a may, for example, be positioned on or integrated with lateral components 420a (for example, flanges) attached to frame 430a of retractor system 400a. In the illustrated embodiment, detectible pattern 414a includes a plurality of spaced lines 416a, which may vary in width. Such detectible components/pattern 414a may, for example, be detectible using electromagnetic energy of various types. For example, light energy may be used in manner similar to a bar code reader. In a number of embodiments, spaced lines 416a are formed of a conductive material such as a metal which may interact with one or more inductive proximity/localization sensors of sensor system 210 positioned on a powered surgical device such as an electrocautery device.

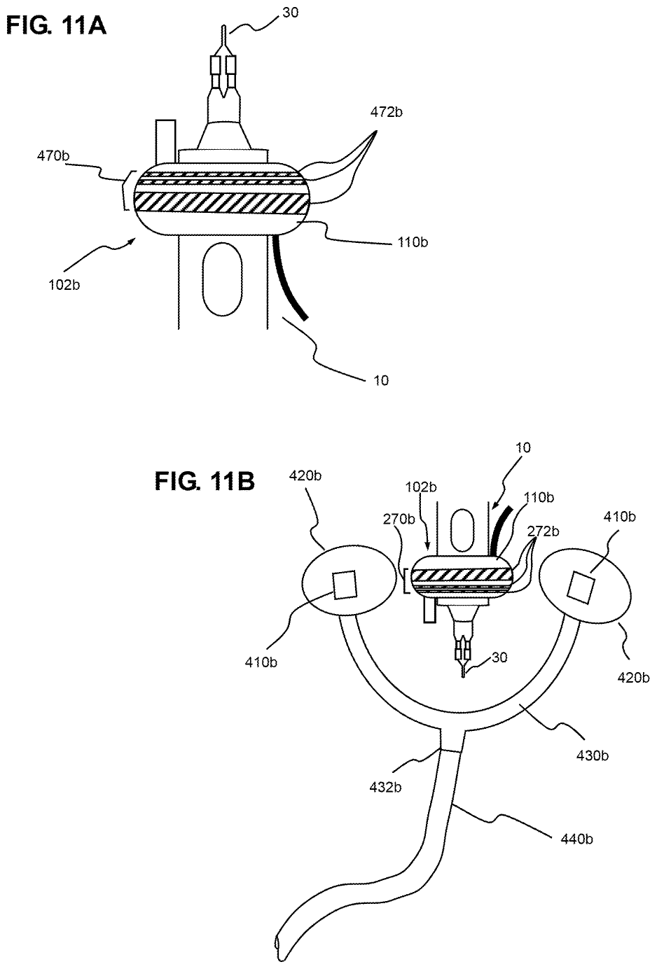

In that regard, FIGS. 10A through 10D illustrate further aspects of the function of mouth retractor system 400a of FIG. 6. FIG. 10A illustrates schematically the positioning of mouth retractor system 400a in operative connection with the mouth of a patient. FIG. 10B illustrates the motion of electrocautery device 10 including a system 100c hereof, which includes two inductive proximity sensors 260c positioned on body 110c of system 102c, relative to one of lateral components 420a and localization pattern 414a thereof. FIGS. 10C and 10D illustrate signals generated as a result of forward and reverse movement, respectively, of proximity sensors 260c past localization patterns 414a. Such pattern/position detection may be analyzed via electronic circuitry 200 to determine both the direction and depth of movement of electrocautery device 10 into the oral cavity. Information on the direction of movement of electrocautery device 10 may be used in an algorithm to control power to electrocautery probe 30. For example, detection of presence and forward motion (into a defined volume or surgical region of interest) as illustrated in FIG. 10C can be used to increase/initiate power to electrocautery probe 30, while detection of reverse or rearward motion (out of a defined volume or surgical regions of interest) as illustrated in FIG. 10D can be used to reduce/cease power to electrocautery probe 30.

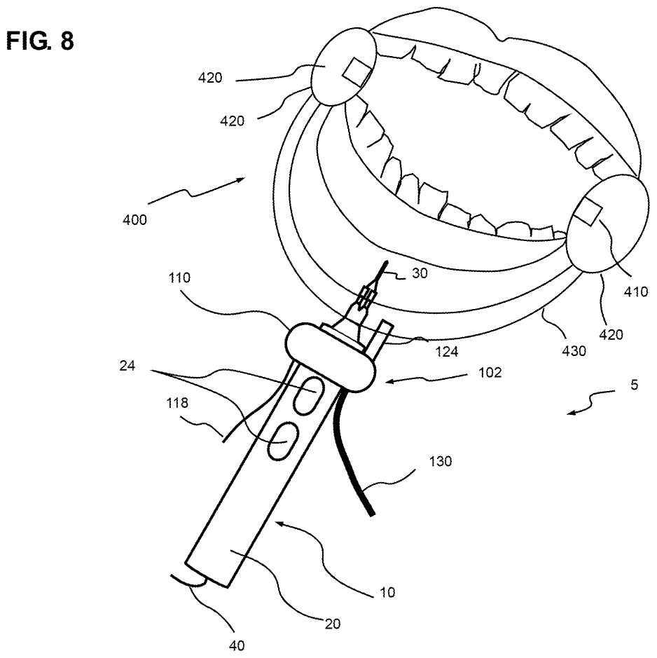

FIG. 7 illustrates another embodiment of a mouth retractor system 400b hereof Mouth retractor system 400b is, in a number of respects, similar in function and design to mouth retractor systems 400 and 400a. Elements or components of mouth retractor system 400b are identified or referenced similarly to like components of mouth retractor systems 400 and 400a with the inclusion of the designation “b” at the end of the reference. Similar to mouth retractor system 400a, mouth retractor system 400b includes components of a localization system which may, for example, be used in inductive proximity sensing. In the case of mouth retractor system 400b, lateral components 420b may include a sensor such as inductive proximity sensors 410b. Wires 412b may, for example, pass through a frame 430b of mouth retractor system 400b, through an end member 432b and into a protective conduit or tube 440b to, for example, place inductive proximity sensor 410b in electrical connection with electrical circuitry 300 or other electrical circuitry.

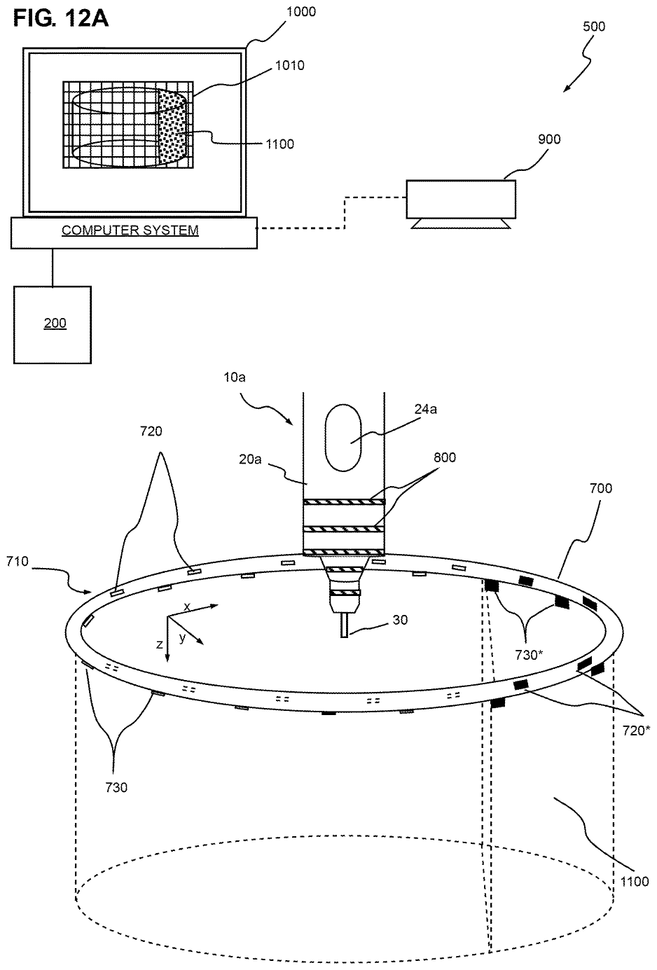

Mouth retractor system 400b may interact with, for example, a system 100b (see, FIGS. 4A, 4B, 11A and 11B) which is in operative connection with an electrocautery device such as electrocautery device 10, in a similar manner as described above in connection with the interaction of system 100c and localization pattern 414a. In that regard, as detectable pattern 470b, which includes a plurality of spaced lines 472b (which may vary in width), a signal/signal pattern is created and analyzed as described in connection with FIGS. 10B through 10C.

Capacitive proximity sensor systems may also be used in the systems hereof. In that regard, a detection apparatus in the form of a frame or boundary (such as a mouth retractor system similar to those describe above) may be placed over or in connection with a body cavity/region of interest. An entrance of the cavity or region of interest may, for example, be contained or defined by the frame or boundary. A signal/source apparatus made of a high capacitance material can, for example, be attached to a powered surgical device such as an electrocautery device (for example, via a system/component such as system 102). A cooperating detection apparatus may, for example, include parallel capacitors plates that provide a signal to a power control module such as electronic circuitry 200 when an object enters or leaves the boundary of a volume defined by the frame. If the signal meets a defined minimal threshold (for example, a high capacitance material entering the region of interest), the power control module will permit power to pass through to the electrocautery device.

In a number of embodiments, a gas-based detection system may be used in localization of an electrocautery probe or an electrocautery device such as electrocautery probe 30 (or another powered tissue-interacting element of a powered surgical device). An attachable apparatus (for example, extending member 120 of system 102 or a separate attachable system) or an integrated component of an electrocautery device may, for example, release a safe/biocompatible and detectible gas such as helium (H) or nitrogen (Na) gas into the operating cavity/region of interest (for example, the mouth) at low rates. Another, separate apparatus may be used to sample the cavity or region of interest to determine whether the gas of interest or target gas (for example, helium or nitrogen) is present. The separate apparatus may include a local sensor for the target gas or include a conduit under negative pressure to transport gas to a remote sensor. Alternatively, an apparatus separate from the powered surgical device may release a target gas. A device on the powered surgical device or on a separate apparatus attachable to the powered surgical device (for example, a sensor or a conduit under negative pressure to transport gas to a remote sensor) may be used to sample the cavity or region of interest to determine whether the gas of interest or target gas is present (for example, at or above a defined threshold concentration). If, for example, the target gas is detected to be present at a defined minimum concentration threshold, a signal can be sent to a power control module such as electronic circuitry 200. As described above, the power control module is connected between the powered surgical device and a power source therefor. In a number of embodiments, as long as a continuous signal is received that the target gas is present at the threshold concentration, the power module will permit power to pass to the powered surgical device to power the tissue-interacting element thereof.

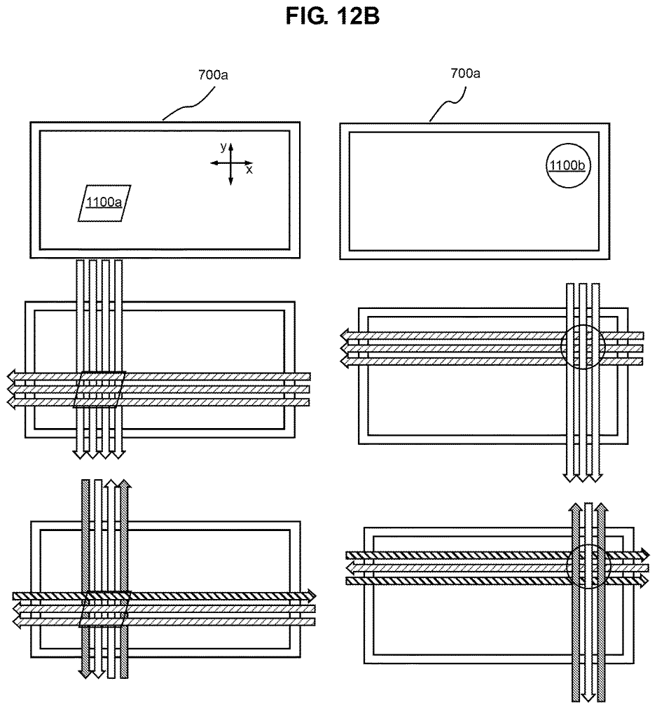

In another system hereof, a signal apparatus in the form of a frame or boundary element is place over or adjacent the entrance to a body cavity or surgical region of interest. The cavity or region of interest may be contained by the frame or boundary. In an embodiment of a system 500 illustrated in FIG. 12A, frame 700 includes an array of energy (for example, infrared energy) emitters and energy (for example, infrared energy) receivers/sensors (or energy/infrared transceivers) as discussed further below. Areas of energy/infrared reflective covering or coverings 800 (for example, IR reflective tape) may be placed on or attached to a powered surgical device 10a including a body 20a. such as electrocautery device 10. As described in connection with device 10, one or more actuators 24a may be included on body 20a which may be held by a user and may include manual control functionality for a surgeon to manually control power/current to a tissue-interacting element 30 on a distal end of electrocautery device 10a. Areas 800 may be present in a pattern to assist in localizing the position of powered tissue-interacting element 30a. Initially, frame 700 provides the ability of an infrared detection system 710 thereof to detect passage of electrocautery device 10a past frame 700 (through the plane of frame 700 or the x-y plane in FIG. 12A) and activate electrocautery device 10 once electrocautery device 10 is within the field or the surgical region of interest and deactivate electrocautery device 10 after its exit from the field. The pattern of reflective areas 800 may assist in determining the position of tissue-interacting element 30a in the z direction.

In the embodiment illustrated schematically in FIG. 12A, system 500 further provides added precision in the surgical field or the surgical region of interest. In the illustrated embodiment, a video camera 900 is positionable over frame 700 and displays a live image of the region of interest on a system monitor 1000. A display of system monitor 1000 may superimpose a grid 1010 over the image via which the user selects which area of the grid is a “safe region 1100”. One or more safe regions/predetermined boundaries and associated volumes may be selected or defined. Infrared sources that are in or define safe region 1100 will be activated or turned on. Because the boundary of interest (as defined by frame 700) will be a 2-dimensional space, infrared sources 720 which emit IR energy in a horizontal direction and infrared sources 730 which emit IR energy in a vertical direction (wherein the horizontal and vertical directions are defined relative to the orientation of the page of the drawing) are attached to frame 700. In FIG. 12A, activated infrared sources are illustrated as enlarged filled rectangles and designate 720* and 730*. To differentiate the two energy directions, control characterizations such as infrared ON-OFF patterns, and/or infrared frequency variation may be used. If electrocautery device 10 is in the safe region 1100, IR reflective material 800 will reflect and disperse the IR energy which will be received by, for example, transceivers 720 and 730 or by one or more separate receivers. The receiver(s) or transceiver 720, 730 will transmit appropriate signals to a power control module (for example, electronic circuitry 200), which may, for example, be connected between the electrocautery device and the power source. If, for example, the power control module detects both characterization signals of the activated horizontal infrared and vertical infrared source, the power control module will permit power to pass through the electrocautery.

Frame 700 may, for example, include one or more extending members which extend in the z-direction (not shown). Energy emitters/sources and energy receivers (sensor) or transceiver may be positioned on such extending member(s). Including emitters/sensors on such extending member(s) can help in localizing or determining the position of tissue-interacting element 30a in the z-direction. Further, images from video camera 900 may be used in localizing the position of tissue-interacting element 30a via, for example, one or more software algorithms stored in electronic circuitry 200.

FIG. 12B illustrates various patterns of energy transmission for detecting presence of a powered surgical device such as electrocautery device 10a. In the embodiments illustrated in FIG. 12B, two-dimensional areas 1100a and 1100b are illustrated in the plane of the opening of or proximal surface defined by frame 700a are intersected by beams of energy transmission represented by arrows. The opening of frame 700a may be used to define the proximal boundary surface of a predetermined volume hereof (that is, the volume extending into the page underneath the two-dimensional areas 1100a and 1100b to a predetermined depth). In the illustrated embodiment, the beams of energy (for example, IR energy) are oriented horizontally and vertically (in the x and y directions) on the page of the drawing. As described above, energy transmitted in a particular direction can be distinguished from energy transmitted in other directions via, for example, differences in modulation sequences and/or differences in frequency, Differences in direction of energy transmitted horizontally (along/parallel to the x axis) and/or vertically (along/parallel to the y axis), which is represented by differences in shading and crosshatching of the energy transmission arrows in the lowermost illustrations in FIG. 12B, can be used to attain additional information regarding the position of the powered surgical device. In a number of embodiments in which the position of the tissue-interacting element further include depth, a sensor/sensor system such as video camera 900 may, for example, be used to determine depth information for the tissue-interacting element of the powered surgical device in embodiment such as those illustrated in FIG. 12B.

Although system 500 may require somewhat more setup time and may be somewhat bulkier that other localization systems described herein, system 500 provides a significant advantages by enabling a user to define specific 3-dimensional volumes or regions as “safe”. System 500 also causes minimal electromagnetic interference, provides suitable, moderate detection distance, is applicable to various cavity sizes, and the resultant signals are suitable to differentiate electrocautery devices from the surrounding environment and random objects.

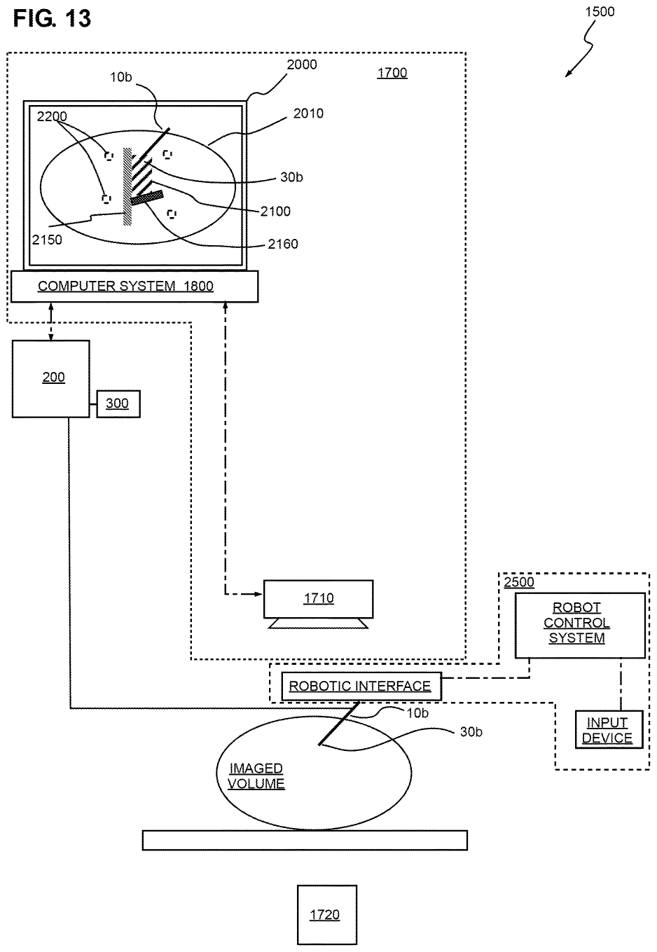

FIG. 13 illustrates another embodiment of a system 1500 hereof in which image guidance (for example, via a computed tomography or CT imaging system or a magnetic resonance imaging (MRI) system) or image guided navigation system is used in localization of a tissue-interacting element 30b of a powered surgical device 10b relative to one or more predetermined boundaries. In general, surgical navigation systems are currently used to guide a surgeon's movements during surgical procedure or operation, An imaging system 1700 displays or visualizes the real-time position of surgical instruments or devices as well as anatomical structure on a display system 2000.

Referring to FIG. 13, as known in the art, CT imaging system 1700 uses an X-ray source 1710 and X-ray detectors, receivers, or sensors 1720. The data from sensors 1720 is used by an associated computer system to produce images of the inside of the body. Such image provide details of any part of the body, including the bones, muscles, fat, organs and blood vessels. Surgical devices such as device 10b also appear on such images during a surgical procedure.

In use of an image-guidance navigation system in a representative example of ENT/sinus surgery, a CT scan of the sinuses is performed using a defined or specified navigation system protocol. A mask, fiducials or markers may be placed on the patients face during the scan to serve as reference points. The CT scan is stored in memory, and then loaded into the image-guidance computer system. During the surgical procedure, a detectable array of fiducials or markers (which may be solid objects or injected fluids) or a mask may be placed on the patient's head. The CT images loaded into the system may be calibrated to the patient's anatomy using set pre-set reference points. Such reference points may, for example, include the mask, fiducials or markers or specific anatomic points on the face. The position of sinus surgical devices (for example, powered surgical device 10c—such as a surgical drill) is tracked by the computer system via integration of the information detected from the patient's pre-set reference points and comparison of that information to the information on the CT scan map.

Such ENT/sinus surgery and other surgeries may, for example, be robotic surgeries. A robotic surgical system 2500 is illustrated schematically in FIG. 13. As described above, in robotic assisted surgical procedures, the surgeon controls one or more robotic interfaces or arms via an input device while at a control center to robotically control surgical device 10b. Similar to non-robotic-assisted surgeries, in robotic surgeries, the surgeon controls the position and movements of powered surgical device 10c as well as the power supplied to a tissue-interacting element 30b of such powered surgical instruments.

Significant injury can arise if tissue-interacting element 30b of powered surgical device 10b is positioned incorrectly and contacts non-targeted tissue such as an artery (for example, the carotid artery) or a nerve. In the case of robotic surgery, control of the procedure is complicated because there is no tactile feedback to the surgeon to, for example, enable tactile discrimination of tissue contact and between contact with different tissue types. Further, if the surgeon accidentally ceases control of the input device, tissue-interacting element 30c may relatively quickly become mispositioned.

The computers system 1800, at least a portion of which may be considered a distributed component of electronic circuitry 200 hereof, of imaging system 1700 may, for example, be configured to identify a predetermined boundary associated with a predetermined volume(s) 2100 (represented schematically with diagonal section lining) as a or safe regions. As with other embodiments hereof, more than once such predetermined boundaries or volumes may be defined. Further, various anatomical structures 2150 and 2160 such as arteries, nerves etc. (represented schematically with shading) throughout the potential path of tissue-contact element 30b during a surgical procedure may be identified a no-go or forbidden regions or be used in defining safe boundaries of one or more predetermined (safe) volumes hereof.

Artificial intelligence/machine learning algorithms (for example, image recognition) may be used in determining various regions or volume hereof. Such algorithms may be combined with technician/surgeon input to attain good precision in defining the regions of volumes. Reference can be made to mask, fiducials or markers represented schematically as broken lined rectangles 2200 on the display of monitor 2000 in FIG. 13.

Allowing surgeons and/or others to predefine volumes or regions which are safe in which to power a tissue-interacting element versus unsafe is a significant advantage, Unintended tissue damage (for example, the lip or facial skin in an oral cautery procedure) is a catastrophic outcome. Use of smart systems hereof in which safe areas/regions are predefined may significantly reduce or eliminate the risk of unintentional tissue damage. The technology hereof may be used in any operating space or body cavity in which surgery is being performed with a powered surgical instrument or device to prevent unintentional injury. In certain surgeries such as electrocautery, additional benefits are provided by combining smart system technology hereof with a sensor system in operative connection with the electrocautery device comprising one or more sensors to detect one or more conditions in the vicinity of a surgical region of interest (for example, including an oxygen sensor). As described above, if the field has an elevated oxygen level, an electrocautery device will not activate, thereby minimizing the risk of an operating room fire.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A system for performing a surgical procedure with a powered surgical device which is configured to be connected to a power source to power a tissue-interacting element of the powered surgical device, comprising:
electronic circuitry configured to be placed in connection with the powered surgical device,
a sensor system in communicative connection with the electronic circuitry, the sensor system configured to measure values of one or more variables related to a risk of injury to non-targeted tissue over time during the surgical procedure, and
the electronic circuitry being configured to control the tissue-interacting element as a function of the measured values of the one or more variables.

2. The system of claim 1 wherein the electronic circuitry is further configured to be placed in communicative connection with the power source and to control the tissue-interacting element via adjustment of power from the power source to the tissue-interacting element.

3. The system of claim 2 wherein the electronic circuitry comprises a processor system and a memory system in communicative connection with the processor system, the memory system having one or more algorithms stored therein, the one or more algorithms being executable by the processor system to analyze the measured values of the one or more variables to determine if one or more thresholds have been met in determining the risk of injury to non-targeted tissue.

4. The system of claim 3 wherein at least one of the one or more variables is related to at least one of (i) a state of an environment in which the powered surgical device is operating during the surgical procedure or (ii) a state of position of the tissue-interacting element of the powered surgical device during the surgical procedure, wherein the state of position comprises one or more of the position of the tissue-interacting element, a rate in change of the position of the tissue-interacting element, a trajectory of the tissue-interacting element, and an orientation of the tissue-interacting element.

5. (canceled)

6. The system of claim 4 wherein the electronic circuity is configured to reduce power to the tissue-interacting element if the position of the tissue-interacting element is determined to be outside of a predetermined boundary.

7. The system of claim 6 wherein the sensor system comprises one or more localization sensors.

8. The system of claim 7 wherein the one or more localization sensors comprise at least one of a proximity sensor, a sensor for a target gas, or an imaging system sensor of an imaging system.

9. The system of claim 7 further comprising a localization system comprising at least one of (i) a frame configured to be positioned adjacent to at least a portion of a surgical region of interest or (ii) one or more fiducials configured to be a reference for an imaging system sensor of an imaging system.

10. The system of claim 9 wherein the frame is a component of a surgical retractor.

11. The system of claim 9 wherein the frame comprises one or more components attached thereto which interact with one or more components on the powered surgical device to localize the position of the tissue-interacting element of the powered surgical device.

12. The system of claim 9 wherein one of the frame or the powered surgical device comprises one or more localization sensors and the other of the frame or the powered surgical device comprises one or more components which interact with the one or more localization sensors.

13. The system of claim 12 wherein the frame has a plurality of energy sources attached thereto and a plurality of energy receivers attached thereto, and wherein the powered surgical device comprises one or more areas of material which is configured to reflect the energy from one or more of the plurality energy sources to the one or more of the plurality of energy receivers.

14. The system of claim 13 wherein the plurality of energy sources emit energy in at least two different directions, wherein energy emitted in each direction is distinguishable from energy emitted in another direction upon reflection to the plurality of energy receivers from the one or more area of material.

15. (canceled)

16. The system of claim 7 wherein the one or more localization sensors comprise one or more imaging system sensors of an imaging system.

17. The system of claim 16 wherein the surgical procedure is a robotic assisted surgical procedure.

18. The system of claim 1 wherein the electronic circuitry is configured to determine if a threshold associated with at least one of the one or more variables has been exceeded.

19. The system of claim 6 wherein the system comprises a selection system via which a user can define the predetermined boundary.

20. The system of claim 1 wherein the powered surgical device is a surgical drill device, a surgical saw device, an electrocautery device, laser emitting device, or a microdebrider device.

21. (canceled)

22. The system of claim 8 wherein the powered surgical device is an electrocautery device, the tissue-interacting element is an electrocautery probe and the frame is a component of an oral surgical retractor.

23. (canceled)

24. (canceled)

25. The system claim 4 wherein at least one of the one or more variables relate to the state of the environment in which the powered surgical device is operating and the sensor system comprises one or more sensors comprising at least one of a temperature sensor or a gas sensor.

26. The system of claim 25 wherein the gas sensor is an oxygen sensor.

27. The system of claim 26 wherein the electronic circuity is configured to reduce power to the tissue-interacting element if a predetermined oxygen concentration threshold is exceeded.

28. The system of claim 26 wherein the electronic circuity is configured to stop power to the tissue-interacting element if a predetermined oxygen concentration threshold is exceeded.

29. The system of claim 25 wherein the powered surgical device is an electrocautery device.

30. (canceled)

31. The system of claim 25 wherein the electronic circuitry is a component of a module configured to be placed in electrical connection with the powered surgical device.

32. A method for performing a surgical procedure with a powered surgical device which is configured to be connected to a power source to powered a tissue-interacting element of the powered surgical device, comprising:
placing electronic circuitry in connection with the powered surgical device,
measuring values of one or more variables related to a risk of injury to non-targeted tissue over time during the surgical procedure via a sensor system in communicative connection with the electronic circuitry, and
controlling the tissue-interacting element as a function of the measured values of the one or more variables.

33.-44. (canceled)
